(19) United States
(12) Patent Application Publication
(10) Pub. No.: US 2024/0002277 A1
(43) Pub. Date: Jan. 4, 2024

(54) RADIOPAQUE GLASS MATERIAL

(71) Applicant: ABK Biomedical Incorporated, Halifax (CA)

(72) Inventors: Sharon LEGERE, Bedford (CA); Daniel BOYD, Upper Tantallon (CA); Kathleen O'CONNELL, Lakeside (CA); Marc GREGOIRE, Stittsville (CA); F. Anthony HEADLEY, JR., Greensboro, GA (US)

(21) Appl. No.: 18/038,933

(22) PCT Filed: Nov. 15, 2021
(86) PCT No.: PCT/CA2021/051616

§ 371 Date: May 25, 2023

Related U.S. Application Data
(60) Provisional application No. 63/120,095, filed on Dec. 1, 2020.

(51) Int. Cl. C03C 3/095; C03C 3/097; C03C 4/08; C03C 3/083

(52) U.S. Cl. CPC C03C 3/095; C03C 3/097; C03C 4/08; C03C 3/083

(57) ABSTRACT

A glass material that includes: from about 0.45 to about 0.86 mole fraction of SiO₂; from about 0.05 to about 0.43 mole fraction of: Y₂O₃, BaO, or a combination of Y₂O₃ and BaO; and optionally Ta₂O₅. The sum of the Y₂O₃, the BaO and the optional Ta₂O₅ is from about 0.10 to about 0.50 mole fraction. The glass includes less than 0.01 mole fraction of Na₂O and less than 0.01 mole fraction of K₂O. The glass material may be in the form of microspheres. The microspheres may be used for vascular embolization and/or radiologic imaging.

### FIELD

The present disclosure relates to radiopaque glass material suitable for forming into microparticles that are administrable to a patient.

### BACKGROUND

The following paragraph is not an admission that anything discussed in it is prior art or part of the knowledge of persons skilled in the art.

Therapeutic vascular occlusions (embolizations) are techniques used to treat certain pathological conditions in situ. Therapeutic embolization is practiced generally using a catheter to position embolization agents in the circulatory system, such as the vessels of various processes: tumors, vascular malformations, and hemorrhagic processes.

### INTRODUCTION

The following introduction is intended to introduce the reader to this specification but not to define any invention. One or more inventions may reside in a combination or sub-combination of the apparatus elements or method steps described below or in other parts of this document. The inventors do not waive or disclaim their rights to any invention or inventions disclosed in this specification merely by not describing such other invention or inventions in the claims.

Particulate embolic agents used in vascular embolization may not be radiopaque, and are therefore difficult to view with radiologic imaging, unless they are treated with a contrast agent prior to injection. TheraSphere™ yttrium-90 microspheres, a commercially available radioactive microsphere used to treat primary and metastatic liver cancer, has a radiopacity of about 6,000 Hounsfield Units (HU) at 120 kVp.

Radiopaque particulate embolic agents are desirable since they may be viewed with radiologic imaging during or after the embolization treatment. One or more examples described in the present disclosure attempt to provide a radiopaque glass material having a radiopacity greater than otherwise comparable particulate embolic agents.

In some applications, glass material that is ultimately intended to be used in particular vascular embolization applications may have a density from about 2.7 g/cm³ to about 4.3 g/cm³.

In the glass material of the present disclosure, Y₂O₃, BaO and Ta₂O₅ all contribute to the radiopacity of the glass. However, increasing the amount of these components also increases the density of the resulting glass material. The authors of the present disclosure have identified glass compositions that provide a desirable amount of radiopacity at densities suitable for vascular embolization. Some glass material of the present disclosure may have a radiopacity of more than 9,000 HU at 120 kVp. Some glass material of the present disclosure may have a density from about 3.5 g/cm³ to about 4.5 g/cm³, such as from about 3.8 g/cm³ to about 4.5 g/cm³. Some glass material of the present disclosure may have a density greater than about 4.5 g/cm³.

In a specific example, a glass composition according to the present disclosure may include: from about 0.59 to about 0.65, such as about 0.62, mole fraction of SiO₂; from about 0.15 to about 0.21, such as about 0.18, mole fraction of Y₂O₃; and from about 0.17 to about 0.23, such as about 0.20, mole fraction of BaO.

In another specific example, a glass composition according to the present disclosure may include: from about 0.52 to about 0.58, such as about 0.55, mole fraction of SiO₂; from about 0.12 to about 0.18, such as about 0.15, mole fraction of BaO; from about 0.07 to about 0.13, such as about 0.10, mole fraction of Ta₂O₅; and from about 0.17 to about 0.23, such as about 0.20, mole fraction of B₂O₃.

In a further specific example, a glass composition according to the present disclosure may include: from about 0.72 to about 0.78, such as about 0.75, mole fraction of SiO₂; from about 0.07 to about 0.13, such as about 0.10, mole fraction of Ta₂O₅; and from about 0.12 to about 0.18, such as about 0.15, mole fraction of Y₂O₃.

In still yet another specific example, a glass composition according to the present disclosure may include: from about 0.79 to about 0.86, such as about 0.83, mole fraction of SiO₂; from about 0.05 to about 0.11, such as about 0.08, mole fraction of BaO; from about 0.06 to about 0.12, such as about 0.09, mole fraction of Ta₂O₅; and from about 0.001 to about 0.006, such as about 0.003, mole fraction of B₂O₃.

In still a further specific example, a glass composition according to the present disclosure may include: from about 0.45 to about 0.55, such as about 0.49, mole fraction of SiO₂; from about 0.19 to about 0.29, such as about 0.24, mole fraction of BaO; from about 0.15 to about 0.25, such as about 0.20, mole fraction of Ta₂O₅; and from about 0.01 to about 0.11, such as about 0.06, mole fraction of B₂O₃.

In yet another specific example, a glass composition according to the present disclosure may include: from about 0.64 to about 0.74, such as about 0.69, mole fraction of SiO₂; from about 0.05 to about 0.15, such as about 0.10, mole fraction of Y₂O₃; and from about 0.16 to about 0.26, such as about 0.21, mole fraction of BaO.

In some examples of the present disclosure, the glass material of the present disclosure is a bulk glass. The term “bulk glass” refers to glass material obtained by forming a glass from the starting reagents without any further processing steps to make the glass material suitable for vascular embolization. For example, glass material produced on a commercial scale without any processing steps to make irregular microparticulate glass material may be considered bulk glass.

In other examples, the glass material of the present disclosure is an irregular microparticulate glass material. The term “irregular microparticulate glass material” refers to particulate material that is sized, but not appropriately shaped, for vascular embolization. Irregular microparticulate glass material may be prepared by pulverizing bulk glass, and sieving the resulting particles to retrieve microparticles of a desired size.

In still other examples, the glass material of the present disclosure is a substantially spherical microparticulate glass material. The term “substantially spherical microparticulate glass material” refers to particulate material that is sized and shaped for vascular embolization. Substantially spherical microparticulate glass material may be prepared by re-melting the surface of irregular microparticulate glass material, and allowing a substantially spherical drop to form.

In another aspect, a method is provided for making glass material according to the present disclosure.

In still another aspect of the present disclosure, substantially spherical microparticulate glass material as described herein may be used for an X-ray based radiologic imaging technique, such as radiography imaging, computerized tomography (CT) imaging, cone beam CT imaging, or fluoroscopy imaging. The present disclosure also provides a method of imaging a mammal using substantially spherical microparticulate glass material as described herein.

In vascular embolization treatments that use radioactive microparticles, it is believed that administering more microparticles with a lower specific activity is desirable because increasing the number of microparticles results in better tumour coverage in comparison to administering fewer microparticles at a higher specific activity. Without wishing to be bound by theory, the authors of the present disclosure believe that microparticles come to rest with the first available localization spots in the vasculature that they encounter. Administering a smaller number of microparticles may concentrate some of the particles in one portion of the tumour if there are sufficient localization spots to interact with a significant portion of the administered particles. In contrast, administering a larger number of lower activity particles better saturates more of the available localization spots and leads to more uniform coverage in the tumour.

For at least some tumor sizes and/or degrees of vascularization, administering to the patient a mixture of (i) radioactive microparticles; and (ii) radiopaque non-radioactive microparticles according to the present disclosure may provide at least some of the benefits associated with administering more microparticles at a lower specific activity, even if the individual radioactive microparticles are at a higher specific activity.

In one aspect, the present disclosure provides a mixture of (i) radioactive glass microparticles; and (ii) non-radioactive, radiopaque microparticulate glass material according to the present disclosure, where the radioactive glass microparticles are suitable to treat tumors in the liver, and where the radioactive glass microparticles and the non-radioactive radiopaque microparticulate glass material have substantially the same size. In particular examples, the microparticulate glass material of the present disclosure and the radioactive glass microparticles have substantially the same density.

In some examples, the mixture may be prepared by exposing pre-radioactive glass microparticles to neutron activation to form the radioactive glass microparticles, and combining the radioactive glass microparticles with the radiopaque microparticulate glass material of the present disclosure.

In still another aspect of the present disclosure, radiation is delivered to a mammal by administering the mixture to the mammal when the mixture includes radioactive glass microspheres. Such a method may additionally include imaging the mammal using an X-ray based radiologic imaging technique, in particular using a static imaging technique. The imaging technique may include fluoroscopy, Computed Tomography/Positron Emission Tomography (CT/PET) or Cone Beam Computed Tomography (CBCT).

Glass material according to the present disclosure may be included in compositions or delivery devices, or used in diagnostic or therapeutic methods.

In some aspects, the present disclosure provides a therapeutic or diagnostic composition that includes a mixture of radioactive microparticles; and non-radioactive microparticles where at least some of the non-radioactive microparticles include the glass material disclosed herein.

In other aspects, the present disclosure provides a method that includes administering the therapeutic or diagnostic composition to a patient, where the administration is: by intravascular delivery, intra-peritoneal delivery, or percutaneous delivery.

In one aspect, the present disclosure provides a delivery device for intravascular delivery, intra-peritoneal delivery, or percutaneous delivery of a mixture of radioactive microparticles and non-radioactive microparticles to a patient. The delivery device is fluidly coupleable to a mixing and transport medium. The delivery device includes a fluid inlet fluidly coupleable to the mixing and transport medium; a fluid outlet; a fluid mixer fluidly coupled to the fluid inlet and to the fluid outlet; a source of radioactive microparticles fluidly coupled to the fluid mixer; and a source of non-radioactive microparticles fluidly coupled to the fluid mixer. At least some of the non-radioactive microparticles are composed of glass material according to the present disclosure. The source of the radioactive microparticles is distinct from the source of non-radioactive microparticles. The fluid mixer mixes the radioactive microparticles with the non-radioactive microparticles, and delivers the mixture of radioactive and non-radioactive microparticles out of the fluid outlet utilizing the mixing and transport medium.

In another aspect, the present disclosure provides a delivery device for intravascular delivery, intra-peritoneal delivery, or percutaneous delivery of a mixture of radioactive microparticles and non-radioactive microparticles to a patient. The delivery device includes: at least one fluid inlet fluidly coupleable to a transport medium; a source of radioactive microparticles fluidly coupled to the at least one fluid inlet; a source of non-radioactive microparticles fluidly coupled to the at least one fluid inlet; a first fluid outlet fluidly coupled to the source of the radioactive microparticles; and a second fluid outlet fluidly coupled to the source of non-radioactive microparticles. At least some of the non-radioactive microparticles are composed of glass material according to the present disclosure. The source of the radioactive microparticles is distinct from the source of non-radioactive microparticles.

In the context of the present disclosure, it should be understood that one population of microparticles is distinct from another population of microparticles if the two populations are not mixed together. For example, radioactive microparticles in the barrel of one syringe would be considered to be distinct from non-radioactive microparticles in the barrel of a second syringe even if the two syringes were fluidly coupled together and capable of expelling the microparticles together to form a mixture.

In still another aspect, the present disclosure provides a method that includes mixing (i) a first population of radioactive microparticles and (ii) a second population of non-radioactive microparticles, and administering a therapeutically or diagnostically relevant amount of the mixture to a patient. At least some of the non-radioactive microparticles are composed of glass material according to the present disclosure.

In yet another aspect, the present disclosure provides a method of administering a therapeutically or diagnostically relevant amount of microparticles to a patient. The method includes: administering non-radioactive microparticles to the patient; and administering radioactive microparticles to the patient without first detecting the non-radioactive microparticles. At least some of the non-radioactive microparticles are composed of glass material according to the present disclosure. The administration is by intravascular delivery, intra-peritoneal delivery, or percutaneous delivery; and the route of administration of the non-radioactive microparticles is the same as the route of administration of the radioactive microparticles.

In yet another aspect, the present disclosure provides a method of administering a therapeutically or diagnostically relevant amount of microparticles to a patient. The method includes: administering radioactive microparticles to the patient; and administering non-radioactive microparticles to the patient without first detecting the radioactive microparticles. At least some of the non-radioactive microparticles are composed of glass material according to the present disclosure. The administration is by intravascular delivery, intra-peritoneal delivery, or percutaneous delivery; and the route of administration of the non-radioactive microparticles is the same as the route of administration of the radioactive microparticles.

In still another aspect, the present disclosure provides a method of administering a therapeutically or diagnostically relevant amount of microparticles. The method includes: concurrent administration of (i) a first population of radioactive microparticles and (ii) a second population of non-radioactive microparticles to a patient. At least some of the non-radioactive microparticles are composed of glass material according to the present disclosure.

In still another aspect, the present disclosure provides a method of administering a therapeutically or diagnostically relevant amount of microparticles. The method includes: sequential administration in a single treatment session of non-radioactive microparticles, and of radioactive microparticles to a patient. At least some of the non-radioactive microparticles are composed of glass material according to the present disclosure.

In yet another aspect, the present disclosure provides a method that includes sequential administration to a patient of (i) therapeutically radioactive microparticles, and then (ii) non-radioactive microparticles. At least some of the non-radioactive microparticles are composed of glass material according to the present disclosure.

In any of the aspects discussed above, the non-radioactive microparticles may be any of the non-radioactive glass compositions discussed in the section below entitled “Glass compositions”; and/or may have any of the features, alone or in combination, of the glass materials discussed in the section below entitled “Glass materials”. In any of the aspects discussed above, the radioactive microparticles may be any of the radioactive glass compositions discussed in the section below related to radiotherapeutic mixtures.

### DETAILED DESCRIPTION

In the context of the present disclosure, it should be understood that “glass material” generally refers to physical material, such as bulk or microparticulate material, that includes glass of the specified composition. The term “glass” or “glass composition” defines the specific components of the composition. Accordingly, reference to physical properties of a material (e.g. particle size) relates to the glass material, while reference to compositional properties (e.g. mole fractions) relates to the glass or glass composition. In some portions of this disclosure, the terms “glass”, “glass composition” and “glass material” are used interchangeably, such as if they all refer to the same component, for example if a glass material is made up only of the noted glass composition.

In the context of the present disclosure, any disclosed range of values should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 1 to about 10” should be interpreted to include not just about 1 to about 10, but also the individual values (e.g., 1, 1.5, 2, 4 . . . etc.) and the sub-ranges (e.g., 1 to 3, 2 to 7, 5 to 6, 2 to 10, etc.) within the disclosed range of values.

Glass compositions. Generally, the present disclosure provides from about 0.45 to about 0.86 mole fraction of SiO₂; from about 0.05 to about 0.43 mole fraction of: Y₂O₃, BaO, or a combination of Y₂O₃ and BaO; and optionally Ta₂O₅. The sum of the Y₂O₃, the BaO and the optional Ta₂O₅ is from about 0.10 to about 0.50 mole fraction. The glass includes less than 0.01 mole fraction of Na₂O and less than 0.01 mole fraction of K₂O.

Glass compositions according to the present disclosure may include: (i) from about 0.50 to about 0.68 mole fraction of SiO₂; (ii) from about 0.10 to about 0.43 mole fraction of: Y₂O₃, BaO, or a combination of Y₂O₃ and BaO; (iii) from about 0.20 to about 0.50 mole fraction of the Y₂O₃, the BaO and the optional Ta₂O₅; (iv) at least some BaO; (v) at least some Ta₂O₅; (vi) at least some Y₂O₃; or (vi) any combination thereof. Some exemplary glass compositions according to the present disclosure, do not include BaO. Some exemplary glass compositions according to the present disclosure do not include Y₂O₃.

Glass compositions according to the present disclosure may include: from about 0.50 to about 0.68 mole fraction of SiO₂; and from about 0.10 to about 0.43 mole fraction of: Y₂O₃, BaO, or a combination of Y₂O₃ and BaO, where the sum of the Y₂O₃, the BaO and the optional Ta₂O₅ is from about 0.20 to about 0.43 mole fraction.

Glass compositions according to the present disclosure may include Ta₂O₅, such as from about 0.05 to about 0.15 mole fraction of Ta₂O₅.

Glass compositions according to the present disclosure may additionally include B₂O₃, such as from about 0.05 to about 0.25 mole fraction of B₂O₃.

Glass compositions according to the present disclosure may consists of, or consists essentially of, the following components: (a) SiO₂; and Y₂O₃, BaO, or a combination of Y₂O₃ and BaO; (b) SiO₂; Y₂O₃, BaO, or a combination of Y₂O₃ and BaO; and Ta₂O₅; (c) SiO₂; Y₂O₃, BaO, or a combination of Y₂O₃ and BaO; and B₂O₃; or (d) SiO₂; Y₂O₃, BaO, or a combination of Y₂O₃ and BaO; Ta₂O₅; and B₂O₃. In the context of the present disclosure, the term “consists of” is synonymous with “consists only of” and refers to a composition that excludes any components that are not expressly recited, but that does not exclude the presence of any impurities in the composition. In the context of the present disclosure, the term “consists essentially of” refers to compositions that include the listed components, plus optionally any unlisted components that (1) do not result in a radiopacity that is less than 9,000 HU at 120 kVp, and (2) do not result in a non-biocompatible particle.

In a specific example, a glass composition according to the present disclosure may include: from about 0.59 to about 0.65, such as about 0.62, mole fraction of SiO₂; from about 0.15 to about 0.21, such as about 0.18, mole fraction of Y₂O₃; and from about 0.17 to about 0.23, such as about 0.20, mole fraction of BaO.

In another specific example, a glass composition according to the present disclosure may include: from about 0.52 to about 0.58, such as about 0.55, mole fraction of SiO₂; from about 0.12 to about 0.18, such as about 0.15, mole fraction of BaO; from about 0.07 to about 0.13, such as about 0.10, mole fraction of Ta₂O₅; and from about 0.17 to about 0.23, such as about 0.20, mole fraction of B₂O₃.

In a further specific example, a glass composition according to the present disclosure may include: from about 0.72 to about 0.78, such as about 0.75, mole fraction of SiO₂; from about 0.07 to about 0.13, such as about 0.10, mole fraction of Ta₂O₅; and from about 0.12 to about 0.18, such as about 0.15, mole fraction of Y₂O₃.

In still yet another specific example, a glass composition according to the present disclosure may include: from about 0.79 to about 0.86, such as about 0.83, mole fraction of SiO₂; from about 0.05 to about 0.11, such as about 0.08, mole fraction of BaO; from about 0.06 to about 0.12, such as about 0.09, mole fraction of Ta₂O₅; and from about 0.001 to about 0.006, such as about 0.003, mole fraction of B₂O₃.

In still a further specific example, a glass composition according to the present disclosure may include: from about 0.45 to about 0.55, such as about 0.49, mole fraction of SiO₂; from about 0.19 to about 0.29, such as about 0.24, mole fraction of BaO; from about 0.15 to about 0.25, such as about 0.20, mole fraction of Ta₂O₅; and from about 0.01 to about 0.11, such as about 0.06, mole fraction of B₂O₃.

In yet another specific example, a glass composition according to the present disclosure may include: from about 0.64 to about 0.74, such as about 0.69, mole fraction of SiO₂; from about 0.05 to about 0.15, such as about 0.10, mole fraction of Y₂O₃; and from about 0.16 to about 0.26, such as about 0.21, mole fraction of BaO.

Glass compositions according to the present disclosure may include substantially no Na₂O and substantially no K₂O. In the context of the present disclosure, the term “substantially no [compound X]” refers to the glass composition not including any compound [X] other than what might be present due to impurities present in the raw materials.

Glass materials. The substantially spherical microparticles according to the present disclosure, which may be useful for embolic vascularization, are produced by first forming a bulk glass. The bulk glass is then processed to provide irregular microparticulate glass material according to the present disclosure. The irregular microparticles are flame treated to form the substantially spherical microspheres. Flame treatment of irregular glass microparticles to form substantially spherical microspheres is well known in the art. Examples of flame treatment include flame spheroidization ultrasonic spray pyrolysis, droplet generator, and vertical thermal flame. Different glass compositions of the present disclosure may melt at different temperatures. Flame treatment processes used to re-melt the surfaces of irregular microparticles may use different gases or gas mixtures, such as a propane-oxygen or acetylene-oxygen, in order to provide a temperature that can re-melt the surface of an irregular glass microparticle of interest. Microparticles that are spheroidized by flame-treatment may be conditioned to reduce or remove surface reaction deposits arising as a result of the flame-treatment.

In the context of the present disclosure, the terms “microparticle” and “microparticulate” may be used interchangeably, and refer to a particle that has a diameter that is less than 1200 μm. For a mixture of particles, the mixture has an average diameter that is less than 1200 μm.

Although the present disclosure may refer to “microspheres” or “glass microspheres” or “spherical particles”, it should be understood that particles of the present disclosure do not need to be perfectly spherical. In the context of the present disclosure, “substantially spherical” refers to a mixture of particles that have an average sphericity (“SPHT”) of at least 0.9. The sphericity (SPHT) may be determined using a CamSizer P4 (ATS Scientific, Burlington, ON) system, operating on dynamic image analysis principle, per ISO IS09276-6 and ISO133322-2, respectively.

SPHT can be determined using the following equation:

$$SPHT = \frac{4 \sqrt{A}}{{P}^{2}}$$

where P is the measured perimeter/circumference of a particle projection and A is the measured area covered by a particle projection; such that an ideal microsphere SPHT is expected to be as 1.0.

The terms “microsphere” and “substantially spherical microparticle” may be used interchangeably and refer to a substantially spherical particle that has an average diameter that is less than 1200 μm. Mixtures of microspheres have an average diameter that is less than 1200 μm.

Bulk glasses according to the present disclosure may have a glass composition as discussed above, which may reflect the theoretical noted mol % of components.

Irregular microparticles may be produced by pulverizing the bulk glass using any technique well known in the art, for example by using a planetary ball mill comprising ZrO₂ grinding media, and sieving the resulting particles to retrieve particulates of a desired size. Using ZrO₂ as a grinding media may help reduce process contaminants due to the toughness of the grinding media relative to the bulk glass.

The irregular microparticles may have an average diameter from about 10 μm to about 1200 μm. Different sized microparticles may be used in different vascular embolization protocols. The microparticles of the present disclosure may be selected to preferentially distribute in tumour vasculature over normal tissue. The size of the microparticles affects this distribution. Microparticles according to the present disclosure, for example that are useful for producing microspheres for visualizing or treating liver tumours, may have average diameters from about 10 μm to about 45 μm. In particular examples, the microparticles may have average diameters from about 10 μm to about 35 μm, or from about 20 μm to about 30 μm. In some examples, irregular microparticles of the present disclosure may be sieved to provide particles from about 20 μm to about 40 μm; from about 20 μm to about 50 μm; from about μm 40 μm to about 500 μm; from about 40 μm to about 300 μm; from about 300 μm to about 500 μm; from about 500 μm to about 700 μm; or from about 700 μm to about 1200 μm. Irregular microparticles of any of these ranges may be used to produced similarly sized microspheres, which may be suitable for one or more vascular embolization protocols. Microspheres obtained from the different sizes of microparticles may be selected depending on the internal diameters of the blood vessels to be occluded. For example, blood vessels that are further from a solid tumour but that still provide blood to support tumour growth may be larger in diameter than blood vessels found within the tumour. It may be desirable to use larger particles to block the larger blood vessels, even if the microspheres are not useful for visualizing the tumour itself.

It should be understood that “about X μm” in the context of particle size and particle diameter is determined based on accepted tolerances as per ASTM E-11 for a test sieve of the noted size. For example, the accepted tolerance for a 50 μm test sieve is 3 μm. Accordingly, “about 50 μm” refers to particles that are from 47 μm to 53 μm in size. In another example, the 15 accepted tolerance for a 35 μm test sieve is 2.6 μm. Accordingly, “about 35 μm” refers to particles that are from 32.4 μm to 38.6 μm in size. The ASTM accepted tolerance for a 25 μm sieve is 2.2 μm. For test sieves without a standard, accepted tolerance (such as test sieves below 20 μm), the expression “about X μm” refers to ±15% for sizes from 5 to 15 μm, and +50% for sizes less than 5 μm. For example “about 1 μm” refers to particles that are from 0.5 20 to 1.5 μm in size.

Irregular microparticles may be flame-treated to re-melt their surfaces and allowing a substantially spherical particle to form. Flame-treating irregular microparticles may be achieved with flame spheroidization by introducing appropriately sized irregular microparticles into a propane/oxygen flame, and directing the flame into a vented collection system. The composition of the irregular glass particles may change when the particles are flame-treated to re-melt the surface of the irregular particles and subsequently allowed to form the substantially spherical droplets.

The optional conditioning of glass microspheres to reduce surface reactivity is well known in the art.

Spherodizing irregular microparticles is not expected to substantially change the diameter of the particles. However, the spheroidized particles may be sieved, either before or after conditioning, to provide particles of the desired size.

Imaging. Radiopaque glass microspheres according to the present disclosure may be used for X-ray based imaging, such as radiography imaging, computerized tomography (CT) imaging, cone beam CT imaging, or fluoroscopy imaging.

A desirable radiopacity of the glass microspheres may depend on the clinical scenario, such as the type of imaging being used, the target treatment area, and/or the estimated packing density of the microspheres. A higher radiopacity glass would be desirable when a relatively small number of microparticles are being delivered, the microparticles are expected to be distributed across a relatively large area, a relatively low-power imaging technique is used, or any combination thereof. Conversely, since too high a radiopacity has the potential to result in imaging artifacts and a decrease in imaging quality, a lower radiopacity glass would be desirably when a relatively larger number of microparticles are being delivered, the microparticles are expected to be distributed across a relatively small area, a relatively high-power imaging technique is used, or any combination thereof.

Glass microspheres according to the present disclosure that are sized to be below 45 μm and that have a density from 3.5 g/cm³ to about 4.5 g/cm³, such as from about 3.8 g/cm³ to about 4.5 g/cm³, may be useful in applications where the microspheres are administered via intra-arterial or intravenous delivery.

Some glass microspheres according to the present disclosure may be compatible with positron emission tomography (PET), single-photon emission computed tomography (SPECT), and/or magnetic resonance imaging (MRI) in that the glass microspheres do not affect the PET, SPECT or MRI imaging.

For imaging applications where the glass microspheres are administered via intra-arterial or intravenous delivery to a patient in order to image the patient's liver, at least about 750 microspheres per gram of liver may be administered to the patient. In some examples, about 1000 to about 5000 microspheres per gram of liver may be administered. For a typical adult human patient, about 1 million to about 7 million microspheres may be administered.

Radiotherapeutic mixtures, compositions, delivery devices, and methods. Radioactive microparticles are manufactured only in a small number of locations, and prepared for delivery to hospitals around the world. The specific activity of the microparticles are calibrated to provide a desired activity at the planned time of administration. For example, TheraSphere, a yttrium-90 glass microparticle, are prepared by neutron activation of yttrium-89 containing glass microparticles to produce microparticles having a nominal specific activity of about 110 GBq/g at the time of calibration, and are typically provided in amounts of about 1.2 million microparticles (about 3 GBq in about 27 mg) to 8 million microparticles (about 20 GBq in about 180 mg) per vial. Depending on the delay between calibration and administration, the amount of activity available to be delivered per vial may range from 0.17 GBq (1.2 million microparticles injected 9 days after calibration) to 18 GBq (8 million microparticles injected 1 day after calibration).

As mentioned above, for a given amount of delivered radioactivity, it is believed that administering more microparticles with a lower specific activity is desirable because increasing the number of microparticles results in better tumour coverage in comparison to administering fewer microparticles at a higher specific activity. For example, in order to administer 3 GBq of radioactivity to a patient, it is believed that administering 6 million microparticles with an overall specific activity of 22 GBq/g results in better tumour coverage than administering 1.5 million microparticles at 88 GBq/g.

Without wishing to be bound by theory, the authors of the present disclosure believe that, for at least some tumor sizes and/or degrees of vascularization, administering to the patient a mixture of (i) radioactive microparticles; and (ii) non-radioactive microparticles according to the present disclosure may provide at least some of the benefits associated with administering more microparticles at a lower specific activity, even if the individual radioactive microparticles are at a higher specific activity.

The present disclosure provides a mixture of (i) radioactive glass microparticles; and (ii) non-radioactive, radiopaque microparticulate glass material according to the present disclosure. The radioactive glass microparticles are suitable to treat tumors in the liver. The radioactive glass microparticles and the non-radioactive radiopaque microparticulate glass material have substantially the same size. Particles that are substantially the same size are expected to behave in substantially the same way after injection into a patient. Accordingly, administering a mixture of particles that are substantially the same size is believed to result in a homogeneous distribution of the radioactive and non-radioactive particles.

In the context of the present disclosure, particles having substantially the same size refers to the average sizes of (a) the radioactive microparticles and (b) the radiopaque, non-radioactive microparticles being within 40% of the average of the two average sizes. For example, the radioactive microparticles may have an average diameter of 20 μm, while the radiopaque, non-radioactive microparticles may have an average diameter of 30 μm. The difference of 10 μm between the two types of microparticles is 40% of the average of the two values. The smaller the size difference, the more similar the particles are expected to behave. Accordingly, it may be preferable for the difference in average sizes to be within 10% of the average of the two averages sizes.

The density of the microparticles may also affect their behavior after injection into a patient. In particular examples, the radiopaque microparticles of the present disclosure and the radioactive glass microparticles have substantially the same density.

The term “particle density” refers to the weight of an individual particle per unit volume. This is in contrast to the term “bulk density”, which refers to the weight of many particles per total volume. Particle density is an intrinsic property of the material, while bulk density will change depending on the properties of the materials in the total volume. Particle density may be discussed in terms of specific gravity, which is the ratio of the density of a substance to the density of a reference substance. In the context of the present disclosure, specific gravity is in reference to water. In the context of the present disclosure, particles having substantially the same density refers to particles that are within about 30%, and preferably within about 15%, of the average.

The particle densities of the (a) radioactive microparticles, and (b) the radiopaque, non-radioactive microparticles may be within about 30%, and preferably within about 15%, of the average. For example, the radioactive microparticles may have a particle density of 3.3 g/cm³, while the radiopaque, non-radioactive microparticles may have a particle density of 4.0 g/cm³. The difference of 0.7 g/cm³ between the two types of microparticles is 19% of the average of the two values.

Mixtures according to the present disclosure may be made using any radiopaque glass composition disclosed herein.

The mixture may be prepared by exposing pre-radioactive glass microparticles to neutron activation to form the radioactive glass microparticles, and combining the radioactive glass microparticles with the radiopaque microparticulate glass material of the present disclosure.

In one particular example, the mixture includes (i) substantially spherical radioactive yttrium oxide-aluminosilicate glass microparticles comprising about 40 wt % SiO₂, about 20 wt % Al₂O₃, and about 40 wt % Y₂O₃ (which is equivalent to about 0.170 mole fraction Y₂O₃, about 0.189 mole fraction Al₂O₃, and about 0.641 mole fraction SiO₂); and (ii) substantially spherical, radiopaque microparticulate glass material according to the present disclosure.

Yttrium-89 may be transformed into yttrium-90 by exposing yttrium-89 containing microparticles to a neutron flux. The specific activity of the resulting microparticles is dependent on the level of flux and the duration of the exposure. For example, yttrium-89 may be exposed to a flux of nominally 10¹⁴ neutrons/cm²/sec to effect neutron activation for a number of days to achieve a specific activity of >150 GBq/g.

An improvement in tumour coverage, for example a more uniform distribution of microparticles, may be achieved with mixtures having radioactive microparticles in an amount from about 80% to about 10% w/w of the total mass of microparticles in the composition. It should be understood that, in the context of the present disclosure, reference to any improvement is in comparison to the same number of radioactive microparticles in the absence of additional non-radioactive microparticles.

With radioactive microparticles having a high specific activity, such as 140 GBq/g, the mixtures may have fewer radioactive microparticles (such as around 10 wt %). In contrast, with radioactive microparticles having a low specific activity, such as 4 GBq/g, the mixtures may have more radioactive microparticles (such as around 80 wt %). In particular examples, such as with radioactive microparticles having a specific activity of about 88 GBq/g, the mixtures may have about 25 wt % radioactive microparticles.

It should be understood that “specific activity” refers to the radioactivity per unit mass of the radioactive microparticles, while “overall specific activity” refers to the radioactivity per unit mass of the mixture of radioactive and non-radioactive microparticles. For example, taking one gram of radioactive microparticles having a specific activity of 10 GBq/g and mixing those microparticles with one gram of non-radioactive microparticles would result in a mixture of microparticles with an overall specific activity of 5 GBq/g.

The mixture of radioactive and non-radioactive particles may be prepared in formulations at a desired radioactivity with different numbers of total microparticles. The total number of microparticles may be selected based on the tumour size and/or degree of vascularization. For example, a formulation having a radioactivity of 10 GBq in 0.5 grams of microparticles may be desirable to treat a tumour with a certain degree of vascularization, while a formulation having a radioactivity of 10 GBq in 1 gram of microparticles may be desirable to treat a more vascularized tumour.

In still another aspect of the present disclosure, radiation is delivered to a mammal by administering a therapeutic amount of the mixture to the mammal when the mixture includes radioactive glass microspheres. Such a method may additionally include imaging the mammal using an X-ray based radiologic imaging technique. Administering to a patient a therapeutic amount of such a mixture of microparticles may allow for the calculation of a delivered dose of radiation to a tissue by non-imagable radioactive microparticles, based on a measured distribution of the non-radioactive, radiopaque microparticles in the tissue.

Without wishing to be bound by theory, the authors of the present disclosure also believe that at least some of the benefits associated with administering a mixture of radioactive and non-radioactive microparticles can be obtained when administering radioactive and non-radioactive microparticles separately.

In some aspects, the present disclosure provides a therapeutic or diagnostic composition comprising a mixture of radioactive microparticles and non-radioactive microparticles, where at least some of the non-radioactive microparticles are composed of the glass material disclosed herein.

The radioactive microparticles and the non-radioactive microparticles may have a difference in particle densities that is within 30%, and preferably within 15%, of the average of the two particle densities.

The radioactive microparticles may have an average diameter from about 10 to about 1200 microns, such as an average diameter from about 20 to about 40 microns. The non-radioactive microparticles may have an average size from about 10 to about 1200 microns, such as an average diameter from about 20 to about 40 microns. The radioactive microparticles and the non-radioactive microparticles may have a difference in average sizes that is within 40% of the average of the two averages sizes.

In some examples, the radioactive microparticles and the non-radioactive microparticles have substantially the same resistance when flowing in a liquid through a conduit.

A skilled person would understand that the resistance of an object flowing in a liquid through a conduit is reflected by the drag coefficient, and that the drag coefficient is a function of skin friction and form drag. Accordingly, resistance of a microparticle flowing in a liquid through a conduit may be affected by, for example: the size, surface area, shape, density of the microparticle, and/or surface condition of the microparticle. A skilled person would also readily understand that two different particles may have substantially the same resistance flowing in a liquid through a conduit since changing a feature to increase drag may be offset by changing another feature to decrease drag. For example, two particles may still have substantially the same drag coefficient, even though the first particle is larger than the second particle, if the surface condition of the first particle is sufficiently smoother than the surface condition of the second particle.

In the context of the current disclosure, the time it takes for a bolus of microparticles to fall a set distance through a liquid may represent the resistance of the microparticles flowing in a liquid through a conduit. This time may be measured by loading a known number of microparticles into a transparent column filled with distilled water. The number of microparticles should be selected so that the height of the bolus of microparticles is from two to five times the inner diameter of the column. Once the microparticles have settled at the bottom of the column, the column is inverted and the microparticles fall through the distilled water, with the drag counteracting the gravitational force. The total time it takes for the microparticles to fall past a transition point is measured. The transition point, measured from the top of the bolus of microparticles, is at least 100 times the inner diameter of the column. For example, in a column with an inner diameter of 0.5 cm, the settled microparticles may be 1.5 cm high, and the total fall time for the bolus of microparticles is the time it takes for all of the microparticles to fall past a point that is 50 cm away from the top of the settled microparticles.

This total fall time is compared to the total fall time for a substantially equal number of a different group of microparticles tested under the same conditions (i.e. the same fluid, the same column, the same transition point). The relative drag ratio is calculated by dividing the fall time for the first group of microparticles by the fall time for the second group of microparticles. In the context of the present disclosure, the first and the second microparticles would be considered to have substantially the same resistance when flowing in a liquid through a conduit if the relative drag ratio was from about 0.95:1 to about 1:0.95.

In some examples, the radioactive microparticles make up from about 10% to about 80%, such as about 25%, of the total mass of microparticles in the composition.

In some examples, the radioactive microparticles are diagnostic radioactive microparticles. In some examples, the radioactive microparticles are therapeutic radioactive microparticles.

Diagnostic radioactive microparticles may include one or more radioisotopes selected from the group consisting of: copper-67, holmium-166, indium-111, iodine-131, lutetium-177, molybdenum-99, phosphorus-32, rubidium-82, technicium-99m, and thallium-201.

Therapeutic radioactive microparticles may include one or more radioisotopes selected from the group consisting of: actinium-225, bismuth-213, copper-67, indium-111, iodine-131, iodine-125, gadolinium-157, holmium-166, lead-212, lutetium-177, palladium-103, phosphorus-32, radium-223, rhenium-186, rhenium-188, samarium-153, strontium-89, and tungsten-188.

The radioactive glass microparticles may be substantially spherical. The non-radioactive microparticles may be substantially spherical.

In another aspect, the present disclosure provides a method that includes administering a mixture of radioactive microparticles and non-radioactive microparticles, as described above, to a patient; where the administration is: by intravascular delivery, intra-peritoneal delivery, or percutaneous delivery.

In a further aspect, the present disclosure provides a delivery device for intravascular delivery, intra-peritoneal delivery, or percutaneous delivery of a mixture of radioactive microparticles and non-radioactive microparticles to a patient. The delivery device is fluidly coupleable to a mixing and transport medium, and includes: a fluid inlet fluidly coupleable to the mixing and transport medium; a fluid outlet; a fluid mixer fluidly coupled to the fluid inlet and to the fluid outlet; a source of radioactive microparticles fluidly coupled to the fluid mixer; and a source of non-radioactive microparticles fluidly coupled to the fluid mixer. The source of the radioactive microparticles is distinct from the source of non-radioactive microparticles. The fluid mixer mixes radioactive microparticles with the non-radioactive microparticles, and delivers the mixture of radioactive and non-radioactive microparticles out of the fluid outlet utilizing the mixing and transport medium. At least some of the non-radioactive microparticles are composed of a glass material according to the present disclosure.

In still another aspect, the present disclosure provides a delivery device for intravascular delivery, intra-peritoneal delivery, or percutaneous delivery of a mixture of radioactive microparticles and non-radioactive microparticles to a patient. The delivery device includes: at least one fluid inlet fluidly coupleable to a transport medium; a source of radioactive microparticles fluidly coupled to the at least one fluid inlet; a source of non-radioactive microparticles fluidly coupled to the at least one fluid inlet; a first fluid outlet fluidly coupled to the source of the radioactive microparticles; and a second fluid outlet fluidly coupled to the source of non-radioactive microparticles. The source of the radioactive microparticles is distinct from the source of non-radioactive microparticles. At least some of the non-radioactive microparticles are composed of a glass material according to the present disclosure. In some examples, the delivery device delivers the radioactive microparticles and the non-radioactive microparticles in a single treatment session. In some examples, the first fluid outlet and the second fluid outlet are proximate to each other. In the context of the present disclosure, it should be understood that the fluid outlets are proximate to each other if the patient could be administered the radioactive microparticles and the non-radioactive microparticles at substantially the same time, for example over the course of a single treatment session.

The radioactive microparticles in the delivery devices may be any radioactive microparticle disclosed herein. The non-radioactive microparticles in the delivery devices may be any non-radioactive microparticle disclosed herein. In some examples, the radioactive microparticles make up from about 10% to about 80%, such as about 25%, of the total mass of microparticles in the delivery device.

In a still further aspect, the present disclosure provides a method that includes mixing (i) a first population of radioactive microparticles and (ii) a second population of non-radioactive microparticles, and administering a therapeutically or diagnostically relevant amount of the mixture to a patient. At least some of the non-radioactive microparticles are composed of a glass material according to the present disclosure. The radioactive microparticles used in the method may be any radioactive microparticle disclosed herein. The non-radioactive microparticles used in the method may be any non-radioactive microparticle disclosed herein. In some examples, the radioactive microparticles make up from about 10% to about 80%, such as about 25%, of the total mass of microparticles used in the method. The administration may be by intravascular delivery, intra-peritoneal delivery, or percutaneous delivery.

In other aspects, the present disclosure provides a method of administering a therapeutically or diagnostically relevant amount of microparticles to a patient. The method includes either: administering non-radioactive microparticles to the patient, and administering radioactive microparticles to the patient without first detecting the non-radioactive microparticles; or administering radioactive microparticles to the patient, and administering non-radioactive microparticles to the patient without first detecting the radioactive microparticles. At least some of the non-radioactive microparticles are composed of a glass material according to the present disclosure. The administration is by intravascular delivery, intra-peritoneal delivery, or percutaneous delivery. The route of administration of the non-radioactive microparticles is the same as the route of administration of the radioactive microparticles.

In some examples, the method includes concurrent administration of the non-radioactive and the radioactive microparticles. In other examples, the method includes sequential administration of the non-radioactive and the radioactive microparticles; or sequential administration of the radioactive and the non-radioactive microparticles.

In still another aspect, the present disclosure provides a method of administering a therapeutically or diagnostically relevant amount of microparticles. The method includes: concurrent administration of (i) a first population of radioactive microparticles and (ii) a second population of non-radioactive microparticles to a patient. At least some of the non-radioactive microparticles are composed of a glass material according to the present disclosure.

In some examples, the first population of radioactive microparticles is distinct from the second population of non-radioactive microparticles. The first population of radioactive microparticles and the second population of non-radioactive microparticles may be administered as a mixture.

In yet another aspect, the present disclosure provides a method of administering a therapeutically or diagnostically relevant amount of microparticles. The method includes sequential administration in a single treatment session of non-radioactive microparticles, and of radioactive microparticles to a patient. At least some of the non-radioactive microparticles are composed of a glass material according to the present disclosure.

In still another aspect, the present disclosure provides a method that includes sequential administration to a patient of (i) therapeutically radioactive microparticles, and then (ii) non-radioactive microparticles. At least some of the non-radioactive microparticles are composed of a glass material according to the present disclosure.

In some examples, sequential administration includes intermittent administration of the non-radioactive microparticles and the radioactive microparticles. The intermittent administration may include alternating administration of the non-radioactive microparticles and the radioactive microparticles.

In some examples, sequential administration includes administration of all of one type of microparticles before administration of the next type of microparticles. For example, sequential administration may include administration of all of the non-radioactive microparticles before administration of any of the radioactive microparticles; or administration of all of the radioactive microparticles before administration of any of the non-radioactive microparticles.

Methods according to the present disclosure may deliver a therapeutically relevant amount of radiation to the patient, or may deliver a diagnostically relevant amount of non-radioactive microparticles to the patient.

In methods according to the present disclosure: the administration may be by intravascular delivery, intra-peritoneal delivery, or percutaneous delivery; the radioactive microparticles and/or the non-radioactive microparticles may be as discussed above; about 10% to about 80%, such as about 25%, of the total mass of microparticles delivered may be radioactive microparticles; or any combination thereof.

Although the above discussion relates to methods of administering radioactive microparticles and non-radioactive microparticles, the present disclosure equally contemplates the corresponding “uses” of the microparticles, including microparticles useful in the disclosed methods, and uses of microparticles in the manufacture of an administrable formulation useful in the disclosed methods.

In the context of the present disclosure, it should be understood that the non-radioactive microparticles discussed in this section of the disclosure may have any of the features, alone or in combination, of the glass materials discussed in the section above entitled “Glass materials”. For example, the non-radioactive microparticles may have any or all of the features associated with microparticles, microspheres, glass microspheres, or spherical particles discussed above.

In the context of the present disclosure, it should be understood that the radioactive microparticles discussed in this section of the disclosure may have any of the features, alone or in combination, of the radioactive glass materials discussed in this disclosure.

### Examples

Irregular Glass Microparticles. The theoretical compositions, based on the relative portions of the starting material, of four exemplary formulations according to the present disclosure are shown in Table 1, below.

TABLE 1
Exemplary formulations according to the present disclosure

| | SiO₂ in | Y₂O₃ in | BaO in | Ta₂O₅ in | B₂O₃ in |
|---|---|---|---|---|---|
| | mol | mol | mol | mol | mol |
| Formulation | fraction | fraction | fraction | fraction | fraction |
| C2 | 0.750 | 0.150 | — | 0.100 | — |
| C5 | 0.550 | — | 0.150 | 0.100 | 0.200 |
| C10 | 0.625 | 0.175 | 0.200 | — | — |
| C13 | 0.832 | — | 0.080 | 0.086 | 0.003 |

For glass synthesis, analytical grade reagents were weighted in accordance with the theoretical compositions outlined in Table 1. The reagents were homogeneously blended for 21 hour prior to subsequent placement in platinum-rhodium crucibles (100 cc to 200 cc). The reagents were exposed to melt parameters at either: (a) 1550° C. for 3 hours, or (b) 1600° C. for 5 hours using an electric furnace (Carbolite Furnaces, Sheffield, UK) and then shock quenched into water. The obtained bulk glass material was dried in an oven (100° C.) overnight.

Combined bulk glass material from various melts were pulverized as a lot using a planetary ball mill comprising ZrO₂ grinding media. The resulting irregular glass microparticulates was sieved to retrieve particulates from 20 μm to 45 μm.

Formulation C2 did not form a glass under either of the tested melt parameters. Formulation C5 formed glass with partial phase separation (glassy appearance), and with low viscosity under tested melt parameters (a) and (b), resulting in glasses referred to as “C5(a)” and “C5(b)”. The formulation C10 tested under melt parameters (a) appeared to have impurities that may have prevented formation of a glass. The formulation C10 formed a glass with little to no phase separation (glassy appearance), and with low viscosity under melt parameters (b), resulting in a glass referred to as “C10(b)”. Formulation C13 did not form a glass under either of the tested melt parameters.

The actual compositions of the produced glass may differ slightly from the theoretical composition. The compositions for glasses C5(b) and C10(b) are reported in Table 2 based on measured values of the corresponding irregular glass microparticles (IGM) sieved to retrieve particulates from 20 μm to 45 μm. These compositions are believed to more accurately reflect the actual composition of the produced glass.

TABLE 2
Measured compositions of exemplary irregular glass microparticles

| | SiO₂ in | Y₂O₃ in | BaO in | Ta₂O₅ in | B₂O₃ in |
|---|---|---|---|---|---|
| | mol | mol | mol | mol | mol |
| Glass | fraction | fraction | fraction | fraction | fraction |
| C5(b) - IGM | 0.54 | — | 0.17 | 0.11 | 0.18 |
| C10(b) - IGM | 0.63 | 0.19 | 0.18 | — | — |

Glass C5(a) had an irregular glass microparticulate density (±SD) of 3.846±0.141 g/cm³ (n=3). Glass C10(b) had an irregular glass microparticulate density (±SD) of 4.064±0.005 g/cm³ (n=3). Glass C5(a) had a microsphere density (±SD) of 4.510±0.003 g/cm³ (n=1). Glass C10(b) had a microsphere density (±SD) of 4.143±0.102 g/cm³ (n=3). The measurements are based on separate samples (n=1 or n=3) from the same lot, but measured as 20 replicates per sample.

X-ray diffraction (XRD) measurements for each composition in irregular glass microparticulate and microsphere form were performed using a Bruker D2 Phaser diffractometer (Bruker AXS Inc., Madison, WI) coupled to an X-ray generator (30 kV; 10 mA) and equipped with a Cu target X-ray tube. Specimens of each experimental material were prepared by pressing the microspheres into hollow zero-background holders. Powder diffraction powders were then acquired in the scan angle range 10°<2θ<60° with a step size of 0.02°.

Microsphere Synthesis. Appropriately classified irregular glass microparticulate can be introduced into a propane/oxygen flame where the flow of oxygen and propane are appropriately controlled. The materials can be re-melted, and spherical liquid droplets can form by surface tension, in a process otherwise known as spheroidization. The flame of the burner can be directed into a stainless-steel collection system which collects the glass microspheres as they are expelled from the flame. The collection system is designed to actively remove any process by-product from the glass microspheres using a water based spay system. The glass microspheres can be subsequently sieved, for example to obtain microspheres with a mean size range from 20 μm to 30 μm.

The composition of the irregular glass particles changes when the particles are flame-treated to re-melt the surface of the irregular particles and subsequently allowed to form the substantially spherical droplets. The compositions of microspheres (MS) formed from glasses C5(b) and C10(b) are reported in Table 3.

TABLE 3
Measured compositions of exemplary glass microspheres

| | SiO₂ in | Y₂O₃ in | BaO in | Ta₂O₅ in | B₂O₃ in |
|---|---|---|---|---|---|
| | mol | mol | mol | mol | mol |
| Glass | fraction | fraction | fraction | fraction | fraction |
| C5(b) - MS | 0.49 | — | 0.24 | 0.20 | 0.06 |
| C10(b) - MS | 0.69 | 0.10 | 0.21 | — | — |

CT Radiopacity. Bulk irregular glass microparticulates and microsphere CT radiopacity was assessed through quantitative radiopacity measurements, expressed as Hounsfield Unit Values (HU) obtained from five replicate regions of interest (ROIs, n=5) recorded from respective Axial CT scans (1 mm slice thickness, pitch=0.5, 70 kVp and 120 kVp) through 3 mL glass v-vials (Product Code: Z115061, Sigma Aldrich, Canada) with 1 g microspheres in 1.8 μL of sterile saline. All measurements were performed on the experimental irregular glass microparticulates, unsieved and sieved to within a mean diameter ranging from 20 μm to 45 μm versus the experimental microspheres within a mean diameter ranging from 20 μm to 30 μm, using a Siemens Somatom Definition AS+ scanner (Siemens Healthcare, Erlangen, Germany) and the extended HU range option employed for scanning.

The CT radiopacity for: unsieved irregular ground microparticulate (NS); irregular glass microparticles (IGM) sieved to obtain particulates from 20 μm to 45 μm; and glass microspheres (MS) sieved to obtain microspheres from 20 μm to 32 μm, are shown in Table 4, below.

TABLE 4
CT Radiopacity values for exemplary glasses
according to the present disclosure

| | CT Radiopacity ±SD, | CT Radiopacity ±SD, |
|---|---|---|
| Glass | HUV at 70 kVp | HUV at 120 kVp |
| C5(a) - NS | 18,546 + 700 (n = 5) | 19,770 + 473 (n = 5) |
| C5(b) - NS | 17,916 + 882 (n = 5) | 19,395 + 374 (n = 5) |
| C10(b) - NS | 18,481 + 988 (n = 5) | 13,217 + 199 (n = 5) |
| C5(b) - IGM | 16,719 + 483 (n = 5) | 17,592 + 337 (n = 5) |
| C10(b) - IGM | 15,110 + 662 (n = 5) | 10,642 + 364 (n = 5) |
| C5(b) - MS | 17,004 + 1108 (n = 5) | 19,618 + 477 (n = 5) |
| C5(b) - MS | 17,047 + 805 (n = 5) | 12,967 + 180 (n = 5) |

Microsphere Conditioning. The flame-treated microspheres can be subjected to a conditioning process post spheroidization, through extraction in calcium and magnesium free phosphate buffered solution (CMF-PBS, Product Code: MT21040CV, Corning™, NY, US) at a ratio of 0.2 g/mL.

The microspheres can be extracted within an enclosed container in a shaking water bath, at 50° C. for 72±2 hours, 120±2 hours, 240±2 hours, 288±2 hours, or for 360±2 hours, under continuous agitation at 120 rpm. Alternatively, the microspheres can be extracted within an enclosed container in a shaking water bath, at 80° C. for 24±2 hours or 72±2 hours, under continuous agitation at 120 rpm.

Post extraction, the microspheres can be separated from CMF-PBS and rinsed (10 times) with sterile water for injection (USP, Ph. Eur. Grade, Rocky Mountain Biologics, MT, US) prior to drying at 120±2° C. until a constant mass (difference in weight 50.1%) was obtained.

The glass microspheres can be stored for analysis or re-sieved, and size sorted to ensure microspheres with a final mean size of 20 μm to 30 μm prior to packaging in cleaned glass storage vials for bulk storage.

Compositional Analysis. For compositional analysis, irregular glass microparticulates and/or microspheres can be subjected to sample preparation by fusion/microwave acid digestion and analyzed by ICP-OES at an IS017025 certified laboratory (NSL Analytical, 4450 Cranwood Pkwy, Warrensville Heights, OH, US) using validated test protocols.

In the preceding description, for purposes of explanation, numerous details are set forth in order to provide a thorough understanding of the examples. However, it will be apparent to one skilled in the art that these specific details are not required. Accordingly, what has been described is merely illustrative of the application of the described examples and numerous modifications and variations are possible in light of the above teachings.

Since the above description provides examples, it will be appreciated that modifications and variations can be effected to the particular examples by those of skill in the art. Accordingly, the scope of the claims should not be limited by the particular examples set forth herein, but should be construed in a manner consistent with the specification as a whole.

What is claimed is:

1. A glass material, wherein the glass comprises:
from about 0.45 to about 0.86 mole fraction of SiO₂;
from about 0.05 to about 0.43 mole fraction of: Y₂O₃, BaO, or a combination of Y₂O₃ and BaO; and
optionally Ta₂O₅,
wherein the sum of the Y₂O₃, the BaO and the optional Ta₂O₅ is from about 0.10 to about 0.50 mole fraction; and
wherein the glass includes less than 0.01 mole fraction of Na₂O and less than 0.01 mole fraction of K₂O.

2. The glass material according to claim 1, wherein the glass comprises:
(i) from about 0.50 to about 0.68 mole fraction of SiO₂;
(ii) from about 0.10 to about 0.43 mole fraction of: Y₂O₃, BaO, or a combination of Y₂O₃ and BaO;
(iii) from about 0.20 to about 0.50 mole fraction of the Y₂O₃, the BaO and the optional Ta₂O₅;
(iv) at least some BaO;
(v) at least some Ta₂O₅;
(vi) at least some Y₂O₃; or
(vi) any combination thereof.

3. The glass material according to claim 1 or 2, wherein the glass comprises:
from about 0.50 to about 0.68 mole fraction of SiO₂; and
from about 0.10 to about 0.43 mole fraction of: Y₂O₃, BaO, or a combination of Y₂O₃ and BaO;
wherein the sum of the Y₂O₃, the BaO and the optional Ta₂O₅ is from about 0.20 to about 0.43 mole fraction.

4. The glass material according to any one of claims 1 to 3, wherein the glass comprises Ta₂O₅, such as from about 0.05 to about 0.15 mole fraction of Ta₂O₅.

5. The glass material according to any one of claims 1 to 4, wherein the glass additionally comprises B₂O₃, such as from about 0.05 to about 0.25 mole fraction of B₂O₃.

6. The glass material according to any one of claims 1 to 5, wherein the glass consists of, or consists essentially of:
(a) SiO₂; and Y₂O₃, BaO, or a combination of Y₂O₃ and BaO;
(b) SiO₂; Y₂O₃, BaO, or a combination of Y₂O₃ and BaO; and Ta₂O₅;
(c) SiO₂; Y₂O₃, BaO, or a combination of Y₂O₃ and BaO; and B₂O₃; or
(d) SiO₂; Y₂O₃, BaO, or a combination of Y₂O₃ and BaO; Ta₂O₅; and B₂O₃.

7. The glass material according to claim 1, wherein the glass comprises:
from about 0.59 to about 0.65, such as about 0.62, mole fraction of SiO₂;
from about 0.15 to about 0.21, such as about 0.18, mole fraction of Y₂O₃; and
from about 0.17 to about 0.23, such as about 0.20, mole fraction of BaO.

8. The glass material according to claim 1, wherein the glass comprises:
from about 0.52 to about 0.58, such as about 0.55, mole fraction of SiO₂;
from about 0.12 to about 0.18, such as about 0.15, mole fraction of BaO;
from about 0.07 to about 0.13, such as about 0.10, mole fraction of Ta₂O₅; and
from about 0.17 to about 0.23, such as about 0.20, mole fraction of B₂O₃.

9. The glass material according to claim 1, wherein the glass comprises:
from about 0.72 to about 0.78, such as about 0.75, mole fraction of SiO₂;
from about 0.07 to about 0.13, such as about 0.10, mole fraction of Ta₂O₅; and
from about 0.12 to about 0.18, such as about 0.15, mole fraction of Y₂O₃.

10. The glass material according to claim 1, wherein the glass comprises:
from about 0.79 to about 0.86, such as about 0.83, mole fraction of SiO₂;
from about 0.05 to about 0.11, such as about 0.08, mole fraction of BaO;
from about 0.06 to about 0.12, such as about 0.09, mole fraction of Ta₂O₅; and
from about 0.001 to about 0.006, such as about 0.003, mole fraction of B₂O₃.

11. The glass material according to claim 1, wherein the glass comprises:
from about 0.49 to about 0.59, such as about 0.54, mole fraction of SiO₂;
from about 0.12 to about 0.22, such as about 0.17, mole fraction of BaO;
from about 0.06 to about 0.16, such as about 0.11, mole fraction of Ta₂O₅; and
from about 0.13 to about 0.23, such as about 0.18, mole fraction of B₂O₃.

12. The glass material according to claim 1, wherein the glass comprises:
from about 0.58 to about 0.68, such as about 0.63, mole fraction of SiO₂;
from about 0.14 to about 0.24, such as about 0.19, mole fraction of Y₂O₃; and
from about 0.13 to about 0.23, such as about 0.18, mole fraction of BaO.

13. The glass material according to claim 1, wherein the glass comprises:
from about 0.45 to about 0.55, such as about 0.49, mole fraction of SiO₂;
from about 0.19 to about 0.29, such as about 0.24, mole fraction of BaO;
from about 0.15 to about 0.25, such as about 0.20, mole fraction of Ta₂O₅; and
from about 0.01 to about 0.11, such as about 0.06, mole fraction of B₂O₃.

14. The glass material according to claim 1, wherein the glass comprises:
from about 0.64 to about 0.74, such as about 0.69, mole fraction of SiO₂;
from about 0.05 to about 0.15, such as about 0.10, mole fraction of Y₂O₃; and
from about 0.16 to about 0.26, such as about 0.21, mole fraction of BaO.

15. The glass material according to any one of claims 1 to 14, wherein the glass includes substantially no Na₂O and substantially no K₂O.

16. The glass material of any one of claims 1 to 15, wherein the glass material is a bulk glass.

17. The glass material of any one of claims 1 to 15, wherein the glass material is an irregular microparticulate glass material.

18. The glass material according to claim 17, wherein the microparticulate glass material has an average diameter from about 10 μm to about 1200 μm.

19. The glass material according to any one of claims 1 to 18, wherein the glass material exhibits a CT Radiopacity of more than 9,000 Hounsfield units at 120 kVp.

20. The glass material according to claim 1 or 19, wherein the density of the glass material is from about 3.5 g/cm³ to about 4.5 g/cm³, such as from about 3.8 g/cm³ to about 4.5 g/cm³, for example from about 3.8 g/cm³ to about 4.2 g/cm³.

21. A microparticulate glass material that is a substantially spherical microparticulate glass material obtained from spheroidizing the irregular microparticulate glass material of claim 17 or 18.

22. The microparticulate glass material of claim 21 wherein spheroidizing the irregular microparticulate glass material comprises re-melting the surface of the irregular microparticulate glass material in a flame, and allowing a substantially spherical drop to form.

23. The glass material of any one of claims 1 to 15, wherein the glass material is a substantially spherical microparticulate glass material.

24. The microparticulate glass material according to any one of claims 21 to 23, wherein the microparticulate glass material has an average diameter from about 10 μm to about 1200 μm.

25. The microparticulate glass material according to claim 24, wherein the microparticulate glass material has an average diameter from about 10 μm to about 1200 μm, such as from about 10 μm to about 35 μm; from about 10 μm to about 45 μm; from about 20 μm to about 30 μm; from about 20 μm to about 40 μm; from about 20 μm to about 50 μm; from about 40 μm to about 500 μm; from about 40 μm to about 300 μm; from about 300 μm to about 500 μm; from about 500 μm to about 700 μm; or from about 700 μm to about 1200 μm.

26. The microparticulate glass material according to any one of claim 21 to 25, wherein the glass material exhibits a CT Radiopacity of more than 9,000 Hounsfield units at 120 kVp.

27. The microparticulate glass material according to any one of claims 21 to 26 for radiography imaging, computerized tomography (CT) imaging, cone beam CT imaging, or fluoroscopy imaging.

28. The microparticulate glass material according to claim 27, wherein the glass material is compatible with positron emission tomography (PET), single-photon emission computed tomography (SPECT), magnetic resonance imaging (MRI), or any combination thereof.

29. A method comprising:
administering to a mammal, via intra-arterial or intravenous delivery, the microparticulate glass material according to any one of claims 21 to 26, and
imaging the mammal, such as the liver of the mammal, by radiography imaging, computerized tomography (CT) imaging, cone beam CT imaging, or fluoroscopy imaging.

30. The method according to claim 29, wherein the mammal is a human.

31. The method according to claim 29 or 30, wherein the method comprises administering at least about 750 particles of the microparticulate glass material per gram of liver, such as about 1000 to about 5000 particles of the microparticulate glass material per gram of liver.

32. The method according to claim 31, wherein the method comprises administering to the human from about 1 million to about 7 million particles of the microparticulate glass material.

33. The method according to any one of claims 29 to 32, wherein the method comprises intra-arterial delivery of the microparticulate glass material into a hepatic artery.

34. Use of the microparticulate glass material according to any one of claims 21 to 26 for imaging a mammal, such as the liver of the mammal, by radiography imaging, computerized tomography (CT) imaging, cone beam CT imaging, or fluoroscopy imaging.

35. The use according to claim 34 for imaging the liver of the mammal, wherein the microparticulate glass material has been formulated to deliver at least about 750 particles of the microparticulate glass material per gram of the liver, such as about 1000 to about 5000 of the particles of the microparticulate glass material per gram of the liver.

36. A mixture of:
the microparticulate glass material according to any one of claims 21 to 26; and
radioactive glass microparticles,
wherein the microparticulate glass material and the radioactive glass microparticles have substantially the same size.

37. The mixture according to claim 36, wherein the difference in average sizes of the microparticulate glass material and the radioactive glass microparticles is within 40% of the average of the two averages sizes.

38. The mixture according to claim 36, wherein the difference in average sizes of the microparticulate glass material and the radioactive glass microparticles is within 10% of the average of the two averages sizes.

39. The mixture according to any one of claims 36 to 38, wherein the microparticulate glass material and the radioactive glass microparticles have substantially the same density.

40. The mixture according to claim 39, wherein the difference in particle densities of the microparticulate glass material and the radioactive glass microparticles is within 30%, and preferably within 15%, of the average of the two particle densities.

41. The mixture according to any one of claims 36 to 40, wherein the radioactive glass microparticles is a yttrium oxide-aluminosilicate glass, preferably comprising about 40 wt % SiO₂, about 20 wt % A12O₃, and about 40 wt % Y₂O₃.

42. The mixture according to any one of claims 36 to 41, wherein the radioactive glass microparticles are substantially spherical.

43. A method for delivering radiation to a mammal, the method comprising: administering to a mammal, via intra-arterial or intravenous delivery, the mixture according to any one of claims 36 to 42.

44. The method according to claim 43, further comprising imaging the mammal, such as the liver of the mammal, by radiography, computerized tomography (CT), cone beam CT, or fluoroscopy.

45. The method according to claim 44, further comprising calculating an absorbed dose of radiation to a tissue.

46. Use of a mixture according to any one of claims 36 to 42 for delivering radiation to a mammal, wherein the mixture is formulated for intravenous or intra-arterial injection to the mammal.

47. A therapeutic or diagnostic composition comprising a mixture of:
(i) radioactive microparticles; and
(ii) non-radioactive microparticles comprising the glass material of any one of claims 1 to 15.

48. The composition according to claim 47, wherein the radioactive microparticles and the non-radioactive microparticles have a difference in particle densities that is within 30%, and preferably within 15%, of the average of the two particle densities.

49. The composition according to claim 47 or 48, wherein the radioactive microparticles have an average diameter from about 10 to about 1200 microns, such as an average diameter from about 20 to about 40 microns; and the non-radioactive microparticles have an average size from about 10 to about 1200 microns, such as an average diameter from about 20 to about 40 microns.

50. The composition according to any one of claims 47 to 49, wherein the radioactive microparticles and the non-radioactive microparticles have a difference in average sizes that is within 40% of the average of the two averages sizes.

51. The composition according to any one of claims 47 to 50, wherein the radioactive microparticles and the non-radioactive microparticles have substantially the same resistance when flowing in a liquid through a conduit.

52. The composition according to any one of claims 47 to 51, wherein the radioactive microparticles make up from about 10% to about 80%, such as about 25%, of the total mass of microparticles in the composition.

53. The composition according to any one of claims 47 to 52, wherein the radioactive microparticles are diagnostic radioactive microparticles.

54. The composition according to any one of claims 47 to 52, wherein the radioactive microparticles are therapeutic radioactive microparticles.

55. The composition according to claim 53, wherein the radioactive microparticles comprise one or more radioisotopes selected from the group consisting of: copper-67, holmium-166, indium-111, iodine-131, lutetium-177, molybdenum-99, phosphorus-32, rubidium-82, technicium-99m, and thallium-201.

56. The composition according to claim 54, wherein the radioactive microparticles comprise one or more radioisotopes selected from the group consisting of: actinium-225, bismuth-213, copper-67, indium-111, iodine-131, iodine-125, gadolinium-157, holmium-166, lead-212, lutetium-177, palladium-103, phosphorus-32, radium-223, rhenium-186, rhenium-188, samarium-153, strontium-89, and tungsten-188.

57. The composition according to any one of claims 47 to 52, wherein the radioactive microparticles comprise about 40 wt % Y₂O₃, about 20 wt % A12O₃, and about 40 wt % SiO₂, wherein at least a portion of the yttrium is yttrium-90.

58. The composition according to any one of claims 47 to 57, wherein the non-radioactive microparticles comprise the glass material according to any one of claims 7 to 14.

59. The composition according to any one of claims 47 to 58, wherein the radioactive microparticles are substantially spherical, the non-radioactive microparticles are substantially spherical, or both.

60. A method comprising:
administering a composition according to any one of claims 47 to 59 to a patient, wherein the administration is: by intravascular delivery, intra-peritoneal delivery, or percutaneous delivery.

61. A delivery device for intravascular delivery, intra-peritoneal delivery, or percutaneous delivery of a mixture of radioactive microparticles and non-radioactive microparticles to a patient, the delivery device being fluidly coupleable to a mixing and transport medium, the delivery device comprising:
a fluid inlet fluidly coupleable to the mixing and transport medium;
a fluid outlet;
a fluid mixer fluidly coupled to the fluid inlet and to the fluid outlet;
a source of radioactive microparticles fluidly coupled to the fluid mixer; and
a source of non-radioactive microparticles fluidly coupled to the fluid mixer, wherein the non-radioactive microparticles comprise the glass material of any one of claims 1 to 15;
wherein the source of the radioactive microparticles is distinct from the source of non-radioactive microparticles; and
wherein the fluid mixer mixes radioactive microparticles with the non-radioactive microparticles, and delivers the mixture of radioactive and non-radioactive microparticles out of the fluid outlet utilizing the mixing and transport medium.

62. A delivery device for intravascular delivery, intra-peritoneal delivery, or percutaneous delivery of a mixture of radioactive microparticles and non-radioactive microparticles to a patient, the delivery device comprising:
at least one fluid inlet fluidly coupleable to a transport medium;
a source of radioactive microparticles fluidly coupled to the at least one fluid inlet;
a source of non-radioactive microparticles fluidly coupled to the at least one fluid inlet, wherein the non-radioactive microparticles comprise the glass material of any one of claims 1 to 15;
a first fluid outlet fluidly coupled to the source of the radioactive microparticles; and
a second fluid outlet fluidly coupled to the source of non-radioactive microparticles;
wherein the source of the radioactive microparticles is distinct from the source of non-radioactive microparticles.

63. The delivery device according to claim 62, wherein the delivery device delivers of the radioactive microparticles and the non-radioactive microparticles in a single treatment session.

64. The delivery device according to claim 62 or 63, wherein the first fluid outlet and the second fluid outlet are proximate to each other.

65. A method comprising:
mixing (i) a first population of radioactive microparticles and (ii) a second population of non-radioactive microparticles, wherein the second population of non-radioactive microparticles comprises microparticles comprising the glass material of any one of claims 1 to 15;
administering a therapeutically or diagnostically relevant amount of the mixture to a patient.

66. The method according to claim 65, wherein the radioactive microparticles make up from about 10% to about 80%, such as about 25%, of the total mass of microparticles in the mixture.

67. The method according to claim 65 or 66, wherein the administration is by intravascular delivery, intra-peritoneal delivery, or percutaneous delivery.

68. A method of administering a therapeutically or diagnostically relevant amount of microparticles to a patient, the method comprising:
administering to the patient non-radioactive microparticles comprising the glass material of any one of claims 1 to 15; and
administering radioactive microparticles to the patient without first detecting the non-radioactive microparticles;
wherein the administration is by intravascular delivery, intra-peritoneal delivery, or percutaneous delivery; and
wherein the route of administration of the non-radioactive microparticles is the same as the route of administration of the radioactive microparticles.

69. The method according to claim 68, wherein the method comprises concurrent administration of the non-radioactive and the radioactive microparticles.

70. The method according to claim 68, wherein the method comprises sequential administration of the non-radioactive and the radioactive microparticles.

71. A method of administering a therapeutically or diagnostically relevant amount of microparticles to a patient, the method comprising:
administering radioactive microparticles to the patient; and
administering to the patient non-radioactive microparticles comprising the glass material of any one of claims 1 to 15, without first detecting the radioactive microparticles;
wherein the administration is by intravascular delivery, intra-peritoneal delivery, or percutaneous delivery; and
wherein the route of administration of the non-radioactive microparticles is the same as the route of administration of the radioactive microparticles.

72. The method according to claim 71, wherein the method comprises concurrent administration of the radioactive and the non-radioactive microparticles.

73. The method according to claim 71, wherein the method comprises sequential administration of the radioactive and the non-radioactive microparticles.

74. A method of administering a therapeutically or diagnostically relevant amount of microparticles, the method comprising:
concurrent administration of (i) a first population of radioactive microparticles and (ii) a second population of non-radioactive microparticles to a patient, wherein the second population of non-radioactive microparticles comprises microparticles comprising the glass material of any one of claims 1 to 15.

75. The method according to claim 74, wherein the first population of radioactive microparticles is distinct from the second population of non-radioactive microparticles.

76. The method according to claim 74, wherein the first population of radioactive microparticles and the second population of non-radioactive microparticles are administered as a mixture.

77. A method of administering a therapeutically or diagnostically relevant amount of microparticles, the method comprising:
sequential administration in a single treatment session of non-radioactive microparticles comprising the glass material of any one of claims 1 to 15, and of radioactive microparticles to a patient.

78. A method comprising:
sequential administration to a patient of
(i) therapeutically radioactive microparticles, and then
(ii) non-radioactive microparticles comprising the glass material of any one of claims 1 to 15.

79. The method according to claim 70, 73, 77 or 78 wherein the sequential administration comprises intermittent administration of the non-radioactive microparticles and the radioactive microparticles.

80. The method according to claim 79, wherein the intermittent administration comprises alternating administration of the non-radioactive microparticles and the radioactive microparticles.

81. The method according to claim 70 or 77, wherein the sequential administration comprises administration of all of the non-radioactive microparticles before administration of any of the radioactive microparticles.

82. The method according to claim 73 or 78, wherein the sequential administration comprises administration of all of the radioactive microparticles before administration of any of the non-radioactive microparticles.

83. The method according to any one of claims 65 to 81, wherein the method delivers a therapeutically relevant amount of radiation to the patient.

84. The method according to any one of claims 65 to 82, wherein the method delivers a diagnostically relevant amount of the non-radioactive microparticles to the patient.

85. The method according to any one of claims 74 to 84, wherein the administration is by intravascular delivery, intra-peritoneal delivery, or percutaneous delivery.

86. The method according to any one of claims 65 to 85, wherein about 10% to about 80%, such as about 25%, of the total mass of microparticles delivered are radioactive microparticles.

87. The mixture according to any one of claims 36 to 42, the composition according to any one of claims 47 to 59, the delivery device according to any one of claims 61 to 64, or the method according to any one of claims 60 and 65 to 86,
wherein the radioactive microparticles and the non-radioactive microparticles have substantially the same resistance when flowing in a liquid through a conduit; and/or
wherein the radioactive microparticles and the non-radioactive microparticles are suitable for vascular embolization.
